(19) United States
(12) Patent Application Publication
(10) Pub. No.: US 2017/0043077 A1
(43) Pub. Date: Feb. 16, 2017

(54) SYSTEM AND METHOD FOR CONTACTLESS POWER TRANSFER IN IMPLANTABLE DEVICES

(71) Applicant: NUHEART AS, Bergen (NO)

(72) Inventors: Vegard TUSETH, Bergen (NO); Eric Rudie, Maple Grove, MN (US); Shawn Patterson, Bergen (NO); Stanley Kluge, Watertown, MN (US); Matthew Keillor, Inver Grove Heights, MN (US)

(21) Appl. No.: 14/825,054

(22) Filed: Aug. 12, 2015

(51) Int. Cl. A61M 1/12

(52) U.S. Cl. CPC A61M 1/127; A61M 1/125; A61M 2210/12

(57) ABSTRACT

In the field of contactless power transfer for medical devices, more specifically, to contactless power transfer for intra-corporeal medical devices, a system and method for transmitting power to a rechargeable power supply are provided, the rechargeable power supply comprising implantable power receiving means, and an implantable power storage means. The system comprises an extra corporeal power transmitting means arranged and configured to supply power to the rechargeable power supply by wirelessly transmitting power to the implantable power receiving means. The system and method are particularly useful in the context of minimally invasive procedures.

### FIELD OF THE INVENTION

The present invention generally belongs to the field of contactless power transfer for medical devices. More specifically, the present invention relates to contactless power transfer for intra-corporeal medical devices. The present invention is particularly useful in the context of minimally invasive procedures, for example those described in PCT application No. PCT/EP2015/055578, entitled ‘PERCUTANEOUS SYSTEM, DEVICES AND METHODS,’ filed 17 Mar. 2015 and expressly incorporated herein by reference in its entirety.

### BACKGROUND

Examples of medical devices that may be suitable for contactless power transfer include implantable medical devices and mechanical circulatory support systems (MCS), for example ventricular assist devices (VADs). A VAD is a mechanical pumping device capable of supporting heart function and blood flow. Specifically, a VAD helps one or both ventricles of the heart to pump blood through the circulatory system. Left ventricular assist devices (LVAD), right ventricular assist devices (RVAD) and biventricular assist devices (BiVAD) are currently available. Circulatory support systems may also include cardiopulmonary support (CPS, ECMO), which provide means for blood oxygenation as well as blood pumping. Such devices may be required during, before and/or after heart surgery or to treat severe heart conditions such as heart failure, cardiopulmonary arrest (CPA), ventricular arrhythmia or cardiogenic shock.

In the field of implantable medical devices and surgery devices, a reduction in the size and weight of these implanted devices is a major benefit for many reasons. For example, the size of an implanted device affects the comfort of a patient. Particularly, a large or bulky implanted device will require more complex surgery and a longer recovery time compared to a small implanted device. An example of such a device is an implantable pacemaker. A pacemaker requires surgery in order to be implant a battery subcutaneously within a patient's body. Such surgical procedures are clearly invasive and unsuitable for weaker and vulnerable patients as they involve a greater recovery time and carry risks of infection and trauma. This is particularly the case in the treatment of children for whom existing surgical equipment and devices are comparatively bulkier and more invasive, and a reduction of the size of the equipment is often difficult, if not impossible, in view of the equipment and procedure involved.

Transcatheter implantation of VADs, for example, involves the insertion through a small incision or puncture made at the groin area of a patient. Existing procedures involve a catheter introduced through an incision adjacent to the groin of the patient and advanced along the femoral vein and inferior vena cava, across the intra-atrial septum and into the left atrium. When required, punctures can be created by known methods using the catheter and the various devices can be inserted through and implanted via the same catheter.

A problem with these types of devices is the incorporation of the battery. A battery can occupy around 50% to 80% of the volume of most implantable medical devices. Furthermore, batteries have a limited lifespan requiring periodic interventions to replace or maintain the batteries. To solve this problem, studies have been carried out on transcutaneous power transmission (non-contact type power transmission). However, problems relating to miniaturisation of the devices and excess heat generation have been reported.

Past efforts using transcutaneous power transfer (magnetic induction) to recharge experimental implanted LVADs were unsuccessful due to the excessive heating of primary and secondary coils. Heating of a coil implanted subcutaneously in the body is undesirable as the surrounding tissue is not a good conductor of heat. This leads to localised heating and tissue damage surrounding the subcutaneous coil. As a result, low powers have to be used in order to prevent excess heating of surrounding tissues. Due to low powers being used, a traditional inductive system requires precise alignment and can only provide power over a small gap. As such, the patient would have to take great care when aligning and positioning an external energy source with the implanted device. Any error in alignment would significantly affect the amount of power being transferred, result in longer charging time, and most significantly, result in excessive heating of the coils.

There are various types of medical devices which may be implanted within the body of a patient and where required, batteries are often integrated within the medical device, leading to a bulky device. Due to their already large size, the choice of implantation location is limited. In addition, such medical devices are often too large for use in certain medical applications and insertion procedures, for example transcatheter insertion procedures.

### SUMMARY OF THE INVENTION

It is an object of this invention to mitigate problems such as those described above.

According to a first aspect of the invention, there is provided a rechargeable power supply for an intra-corporeal medical device comprising an implantable means for wirelessly receiving power and an implantable power storage means.

The rechargeable power supply according to the present invention reduces the need for a large implantable power storage means. The power storage means can be wirelessly charged and, therefore, the size of the power storage means can be significantly reduced. This reduces the complexity of the surgery required to implant the power storage means and/or the medical device as the elements are substantially smaller. The rechargeable power supply can be arranged and configured to be implanted within the circulatory system via, for example, percutaneous and/or transcatheter methods.

Preferably, the rechargeable power supply is arranged and configured to be implanted within the intravascular and or cardiovascular system of a patient, for example a vein, artery or vena cava. Implantation utilising the intravascular system results in less traumatic procedures because fewer puncture/incision sites are required. For example, the rechargeable power supply and the intra-corporeal device to be charged may be implanted via a single puncture/incision site, and moved into position via the vascular system as a means of transportation, thereby by-passing internal anatomical walls.

The present invention is particularly advantageous when used in conjunction with transcatheter medical devices, as both the medical device and the power supply can be inserted through and positioned in the same anatomical space, e.g. in the cardiovascular system. Consequently, the risk of cross-contamination between anatomical spaces and infection is reduced.

By contrast, subcutaneous batteries will require a separate procedure to be positioned in a site separate from that of the medical device and further procedures are required to allow interconnection of the battery. There is therefore a risk of infection to surrounding tissues at the insertion site, at the interconnection sites and at the implantation sites. Where the medical device is implanted in an anatomical space other than that of the power supply, there is a risk of cross-contamination between the anatomical spaces. In the case of the vascular system, accidental blood loss can be lethal.

Within the context of this invention, the expressions “anatomical space” or “anatomical compartment” can be used interchangeably and may be the vascular system, the cardio vascular system, the gastric system, the respiratory system or other anatomical compartments.

Within the context of this invention, the expressions “percutaneous” and “transcatheter” methods can be used interchangeably and refer to methods involving a procedure carried out through or via a tubing or catheter positioned in the patient's body.

Within the context of this invention, the expression “intra-corporeal” means inside the patient's body and “extra-corporeal” means outside the patient's body.

Furthermore, an advantage may be that the rechargeable power supply, which may consist of a coil and a battery, may not need to be recovered for maintenance purposes. For example, the battery can be charged whilst still implanted in the body and can remain for a semi-permanent basis, until for example the device is no longer required or a defect needs to be fixed.

Preferably, the implantable power receiving means is arranged and configured to receive power from an extra-corporeal means for wirelessly transmitting power. An advantage of this feature is that the implantable means of the rechargeable power supply can be charged without removal of these elements.

Preferably, the implantable power receiving means further comprises implantable means for supplying the power received by the power receiving means to the power storage means. An advantage of this feature is that the power can be configured prior to receipt by the power storage means, for example an AC-DC converter.

Preferably, the implantable power receiving means is arranged and configured to receive power in the form of magnetic flux. An advantage of this feature is that the magnetic flux can harmlessly propagate through the body of a patient, allowing for contactless power transfer.

Preferably, the implantable power receiving means and an extra-corporeal means for wirelessly receiving power are positioned to enable magnetic coupling, thereby enabling power transmission between the extra-corporeal power transmitting means and the implantable power receiving means. An advantage of this feature is that correct alignment increases coupling between the different means, allowing more efficient and quicker power transfer between the means. This may also reduce undesirable heating of the means during power transfer.

Preferably, the implantable power receiving means comprises an electromagnetic coil. The coil may be concentrically wound (i.e. a planar or flat coil) or be helical (i.e. a solenoid). In a preferable embodiment, the coil may comprise a solenoid. An advantage of a solenoid is that it can be implanted in the vascular system through a catheter, i.e. without requiring surgery. In addition, a solenoid can be implanted in an area less exposed to impacts (for example in the cardiovascular system), thus reducing the chances of damage from impacts to the patient's body. Indeed, most planar coils are implanted subcutaneously, so that they can follow the shape of the patient's skin surface. However, subcutaneous coils are more exposed to impact and, in view of their shape, more likely to become deformed due to impact or even just physical movement. If the shape of the coil becomes distorted, then the efficiency of the power receiving means will become affected.

Another important advantage of a power receiving means comprising a solenoid is that the solenoid can dissipate heat more efficiently compared to a planar device. This is particularly true when the power receiving means is positioned where more of its surface area is in contact with bodily fluids. For example, when the power receiving means is positioned in the vascular system, the patient's blood can cool it down and there is no risk of overheating owing to the patient's blood flow which dissipates the heat. Thus a solenoid may not require a cooling system in order to operate safely and efficiently.

Preferably, the longitudinal axis of the electromagnetic coil is arranged and configured to be substantially parallel to the longitudinal axis of an extra-corporeal power transmitting means, more preferably coaxial. An advantage of this feature is that rotation of the electromagnetic coil around its longitudinal axis will not affect the coupling efficiency of the electromagnetic coil with respect to the extra-corporeal power transmitting means. This is particularly advantageous when the longitudinal axis of the coil is positioned vertically with respect to an anatomically vertical portion of a vein or artery, for example a portion of the vena cava.

Within the context of this invention, the expression “vertical” is used relative to the human body, i.e. in the head-feet direction.

Preferably, the rechargeable power supply comprises an extra-corporeal power transmitting means, which will be described in more detail below.

Preferably, the implantable power receiving means and the extra-corporeal power transmitting means operate at substantially the same resonant frequency. An advantage of this feature is that the wireless resonant power transfer increases the coupling distance between the implantable power receiving means and the extra-corporeal power transmitting means, reduces alignment and orientation related coupling issues, and reduces heating of the different means.

Preferably, the implantable power receiving means and the extra-corporeal power transmitting means are arranged and configured to be capacitively loaded to form a tuned LC circuit.

Preferably, the size, shape and dimensions of the implantable storage means and/or the implantable power receiving means are such that they can be implanted easily in different parts of the body without accidentally affecting bodily functions. More preferably, each or both are substantially elongated. For example, the elongated means may be implanted within the circulatory system, e.g. a vein or artery, without impeding fluid flow.

Preferably, the implantable power receiving means and/or the implantable power storage means are each or both arranged and configured to be implanted within the circulatory system, preferably in a vein or an artery, more preferably within the inferior vena cava. An advantage of the power receiving means and/or the implantable storage means being implanted within the circulatory system may be that heat generated from these devices can be dissipated more efficiently. For example, fluid flow within the circulatory system acts as a cooling system that dissipates heat, preventing a build up of heat at the location of these devices. In some cases, the heat transfer capability of the circulatory system may be 1000 times higher than that of subcutaneous tissue. Thus an electromagnetic receiving coil implanted within the circulatory system may be able to receive much higher power compared to a subcutaneous device. For example, it is envisaged that powers of up to 20 watts or greater could be utilised without excess heating of the flowing fluid within the circulatory system. This may allow higher powers to be used during charging and, thus, speed up the power transfer. Furthermore, these means stay in the same intra-corporeal space, and are less prone to dislocation/damage from impacts to the patient's body compared to subcutaneous devices close to the surface of the skin. In subcutaneous devices, there is a risk of burning or injury to the patient, as the body insulates any heat generated from these devices and prevents heat dissipation, resulting in localised heating/burning of tissues. Thus implanting a receiving means, for example an electromagnetic coil, within the cardiovascular system via surgery or other means may allow higher powers to be used during contactless power transfer. Further, a larger receiving means, for example electromagnetic coil, can be utilised in the cardiovascular system compared to subcutaneous devices. This may be because the vessels of the cardiovascular system are relatively long, allowing for a potential electromagnetic coil to range in size from a few millimetres to over thirty centimetres. Similarly, the diameter of the electromagnetic coil and/or rechargeable battery, for example, may be in the range of 1-20 mm. More preferably, the diameters may be in the range of 5-10 mm, obviously depending on the area of the cardiovascular system being utilised. The increased size of the electromagnetic coils compared to the prior art, coupled with an efficient cooling mechanism in the form of the cardiovascular fluid flow, mean that the efficiency and speed of power transfer between a transmitting and receiving coil is increased. This may result in a larger tolerance when aligning the receiving and transmitting coils, resulting in a simpler method of power transfer for the patient.

Preferably, the implantable storage means comprises a rechargeable battery. This has an advantage of negating the need for a permanent power supply being in communication with the implantable storage means, for example a battery belt.

Preferably, the implantable power storage means is separate from the implantable power receiving means. An advantage of this feature is that a modular design may allow for easier maintenance of the different means, allow for easier implantation of the different means, and allow for optimum positioning of each of the means, which may depend on shape of the implanted location, need, and coupling efficiency. Furthermore, the modular design may allow for one or more of the elements to be easily replaced if and when required. For example, some VADs are destined for permanent or semi-permanent implantation, but the battery may require replacing. Advantageously, the modular elements may allow for specific tailoring of implant locations for specific patients, for example patients with anatomical defects or injury. Therefore, the modular design may provide for a more versatile implementation compared to current devices.

Preferably, the implantable power storage means is integrated with the implanted power receiving means. An advantage of this feature is that of compactness. Furthermore, in this configuration, the power receiving means may be less prone to damage from impacts, since it may be supported by the power storage means. Further, there may be less power loss due to a shorter interconnection being required between the power storage means and the power receiving means.

Preferably, the rechargeable power supply is made partially or completely from a bio-compatible material and/or medical grade material.

According to a second aspect of the invention, there is provided an intra-corporeal medical device comprising a rechargeable power supply, the rechargeable power supply comprising means for wirelessly receiving power and power storage means.

The intra-corporeal medical device according to the present invention can be made smaller than prior art devices due to a smaller battery being required. The medical device can be wirelessly charged and, therefore, the battery can be made smaller.

Preferably, the power receiving means and/or the power storage means are each or both integrated within the medical device. This has the advantage of compactness, and only requiring a single procedure to implant the device, which would lead to less invasive procedures for the patient. Furthermore, integration may have an advantage of reducing corrosion of the power receiving means, and preventing the power storage means from leaking into the body.

Preferably, the power receiving means and/or the power storage means are each or both separate from the medical device. An advantage of this feature is that a modular design may allow for easier maintenance of the different means, allowing for easier implantation of the different means and allow for optimum positioning of each of the means, which may depend on shape of the implanted location, need, and coupling efficiency. Furthermore, the modular design may allow for one or more of the elements to be easily replaced when required. Advantageously, the modular elements may allow for specific tailoring of implant locations for specific patients, for example patients with anatomical defects. Therefore, the modular design may provide for a more versatile implementation compared to current devices.

Preferably, the intra-corporeal medical device further comprises implantable means for supplying power received by the power receiving means to the power storage means. More preferably, the means for supplying power is a power converter, preferably an AC-DC converter.

According to a third aspect of the invention, there is provided an implantable wireless power receiving device for a rechargeable power supply as described above.

According to a fourth aspect of the invention, there is provided an implantable power storage device for a rechargeable power supply as described above.

According to a fifth aspect of the invention, there is provided an extra-corporeal power transmitting device arranged and configured to supply power to a rechargeable power supply, comprising means for wirelessly transmitting power, wherein the power transmitting means is arranged and configured to transmit power to an implanted power receiving means.

Preferably, the power transmitting means comprises an electromagnetic coil.

Preferably, the power transmitting means and an implantable power receiving means are aligned to facilitate magnetic coupling, thereby enabling power transmission between the extra-corporeal power transmitting means and the implantable power receiving means.

Preferably, the longitudinal axis of the electromagnetic coil is arranged and configured to be substantially parallel to the longitudinal axis of an implantable power receiving means.

Preferably, the extra-corporeal power transmitting device further comprises means for positioning the power transmitting means relative to the implanted power receiving means to enable magnetic coupling. This may have an advantage of facilitating efficient magnetic coupling, reducing the time required for charging the implanted power receiving means, and reducing heat generated by the implanted power receiving means and the extra-corporeal power transmitting device.

Preferably, the positioning means, in use, is positioned around the torso of the patient. More preferably, the positioning means, in use, is positioned around the abdomen of the patient. Alternatively, one could envisage a positioning means which, in use, is positioned around one of the patient's limbs. For example, the rechargeable power supply may be positioned in the arm of the patient, and the extracorporeal power transmitting means may be positioned around the arm.

Preferably, the positioning means is substantially tubular, more preferably a wearable garment, more preferably an arm band, leg band, vest and/or belt.

Preferably, the power transmitting means and/or the positioning means are each or both extendible. I.e. each or both means may comprise or consist of an extendible material. This may have an advantage of allowing the device to form around the body of the patient without moving/slipping off the patient, which could otherwise affect the alignment of the device.

According to a sixth aspect of the invention, there is provided a method for supplying power to an intra-corporeal medical device, comprising the step of implanting a rechargeable power supply in a patient.

Preferably, the method further comprises the step of wirelessly transmitting power from an extra-corporeal power transmitting device to the implanted power receiving means.

Preferably, the method further comprises the step of supplying the power received by the power receiving means to the power storage means.

Preferably, the power receiving device and/or the power storage means are, in use, positioned in the circulatory system, preferably in a vein or an artery, more preferably within the inferior vena cava.

Preferably, the method for supplying power to the intra-corporal medical device may be performed by percutaneous or transcatheter procedures.

### BRIEF DESCRIPTION OF THE DRAWINGS

The invention will be further described with reference to the drawings and figures, in which:

FIG. 1 is a schematic representation of a rechargeable power supply system for an intra-corporeal medical device implanted within the body of a patient according to aspects of the present invention;

FIG. 2 is a schematic representation of a further representation of a rechargeable power supply for an intra-corporeal medical device according to aspects of the present invention;

FIG. 3 is a simplified block diagram illustrating functional blocks of a rechargeable power supply for an intra-corporeal medical device according to aspects of the present invention;

FIG. 4 is a simplified block diagram illustrating functional blocks of an extra-corporeal power supply for charging the rechargeable power supply in FIGS. 1-3 according to aspects of the present invention;

FIG. 5 is a schematic representation of an intra-corporeal medical device comprising a rechargeable power supply according to aspects of the present invention;

FIG. 6 is a flow chart illustrating steps for supplying power to an intra-corporeal medical device according to aspects of the present invention;

FIG. 7 is a schematic representation of an extra-corporeal power supply for charging the rechargeable power supply according to aspects of the present invention; and

FIGS. 8a and 8b are schematic representations of an alternative rechargeable power system for an intra-corporeal medical device implanted within the body of a patient according to aspects of the present invention.

### DETAILED DESCRIPTION

The invention is described by way of examples, which are provided for illustrative purposed only. These examples should not be construed as intending to limit the scope of protection that is defined in the claims. For example, although various aspects have been described with respect to the heart and the circulatory system, this is not intended to be limiting, and is merely performed to provide an example implementation. Aspects disclosed herein may be utilised in any medical device implantable within the human body, for example in the cardiovascular system, respiratory system, gastric system, neurological system and the like, some examples including neurostimulators, implantable defibrillators, and pacemakers, implantable drug-delivery pumps. As used herein, the term “means” can be equivalently expressed as, or substituted with, any of the following terms: device, apparatus, unit, structure, part, sub-part, assembly, sub-assembly, machine, mechanism, article, medium, material, appliance, equipment, system, body, or similar wording.

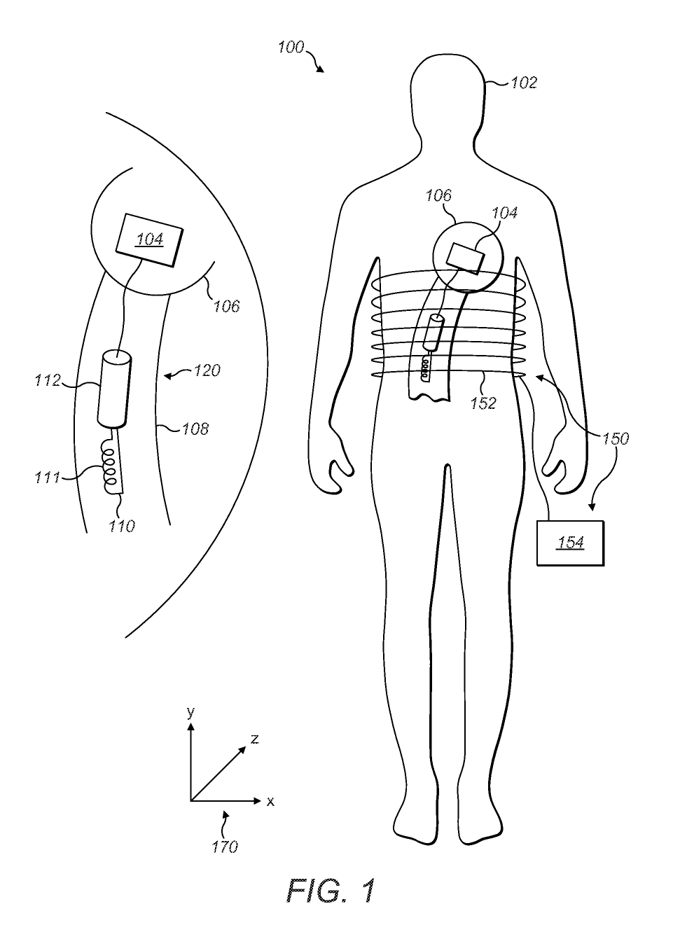

Referring to FIG. 1, there is illustrated a system 100 including a rechargeable power supply 120 for an intra-corporeal medical device 104, comprising implantable means 110 for wirelessly receiving power and implantable power storage means 112. System 100 further comprises an extra-corporeal power supply 150. In this specific embodiment, the intra-corporeal medical device 104 is implanted within the heart 106 of the patient 102. Specifically, the medical device 104 connects the left atrium to the aorta of the patient's 102 heart 106. The rechargeable power supply 120 is situated within part of the patient's 102 circulatory system, for example the patient's vena cava 108. The circulatory system may include for example the left atrium, the right atrium, the left ventricle, the right ventricle, the aorta, the vena cava as well as arteries, veins and other compartments of the peripheral vascular system. The rechargeable power supply 120 comprises an implanted module 110 for wirelessly receiving power (e.g. a coil), and a power storage module 112 (e.g. a rechargeable battery). The implanted module 110 is operably coupled to the power storage module 112 (e.g. via a medically suitable electrical connector), and the power storage module 112 is further operably coupled to the intra-corporeal medical device 104. In this specification, the term ‘operably coupled’ is a term used to describe a link between two or more components. The term can define a physical link between components, for example an electrical connector, or a wireless link between components, for example by inductive coupling.

In this embodiment, the medical device 104 may be a mechanical circulatory support system (MCS), for example a ventricular assist device (VAD), which requires constant power in order to assist blood flow. The power storage module 112, which may be a type of rechargeable storage device such as a rechargeable battery, is arranged and configured to supply power to the intra-corporeal medical device 104. The power storage module 112 may be cylindrical in shape and have dimensions suitable for allowing the device to be positioned within the circulatory system without inhibiting fluid flow within the circulatory system. For example, the power storage module 112 may have a diameter in the range of 1-20 mm, more preferably the power storage module 112 may have a diameter in the range of 5-10 mm. The implanted module 110 is arranged to wirelessly receive power via for example a coil, from the extra-corporeal power supply 150 and couple this power to the power storage module 112 via for example a suitable cable and connector, thereby recharging or maintaining charge within the power storage module 112. In this example, the coil may have dimensions similar to that of the power storage module 112, so that it can be positioned within the circulatory system without inhibiting fluid flow. Furthermore, the coil may be designed to have a longitudinal direction arranged coaxially with the vein or artery that it is implanted within. In some example, the coil may have a length up to or in excess of 30 centimetres, depending on the area of the cardiovascular system being used.

In this embodiment, the extra-corporeal power supply 150 comprises a coil 152, for example an electromagnetic coil, electrically couplable to a power source 154. The coil 152 is arranged and configured around the exterior of the patient 102, wherein the windings of the coil 152 are positioned such that they substantially coil around the torso of the patient 102. The windings of the coil may be positioned in close proximity to each other or be positioned as illustrated with respect to coil 152, wherein the coil has a longitudinal direction with respect to the patient 102. The implanted module 110 may also comprise a coil 111 that is operably coupled to the power storage module 112. The coil 111 is also implanted within the vena cava 108 of the patient 102. The coil 111 of the implanted module 110 also comprises windings, which are arranged in a coaxial position relative to the windings of the coil 152 of the extra-corporeal power supply 150. This has an advantage of allowing efficient magnetic coupling between the coils.

In an example operation, the power source 154 of the extra-corporeal power supply 150 couples power to the coil 152. The coil 152 will subsequently produce a magnetic field that is strong enough to be coupled to the coil 111 of the implanted module 110, which is positioned distal from the coil 152. In this example, magnetic flux generated by the coil 152 will be produced in the ‘y’ direction of axis 170. The coil 111 is arranged and configured to receive the magnetically coupled power from the coil 152, and supplies this power to the power storage module 112. This is achieved by aligning the coil 111 so that it is parallel to the coil 154, for example the coil 111 is arranged so that it couples the magnetic flux generated in the ‘y’ direction according to axis 170. Therefore, non-radiative energy can be transferred between power supply 154 and power storage module 112 in the form of coupled magnetic flux, via coils 111 and 152. This has an advantage of allowing the power storage module 112 implanted within the vena cava 108 of the patient 102 to be charged without the need for surgical intervention.

It should be noted that in this example embodiment, the rechargeable power supply 120 comprises modular components in the form of a modular implanted module 110 and a modular power storage means 112, for example a cylindrical rechargeable battery and a solenoid. Providing a modular system may allow for the various components of the rechargeable power supply 120 to be maintained without requiring surgical removal of the entire rechargeable power supply 120 or the medical device 104. For example, each modular portion of the rechargeable power supply 120 may be implanted or removed via percutaneous means, thereby simplifying the procedure, reducing patient recovery times and reducing the chance of infection. Furthermore, the modular design of the rechargeable power supply 120 may allow it to be situated within small areas, such as veins, arteries or the inferior vena cava, whilst still facilitating blood flow in those areas. Positioning the rechargeable power supply 120 within the intravascular system allows for greater flexibility in terms of positioning. Further, unlike subcutaneous devices, positioning the rechargeable power supply 120 within the intravascular system may make it less prone to damage from impacts to the patient's body.

In an example, the solenoid and the rechargeable battery may be maintained in their desired position via a cable that electrically connects them to each other and/or the medical device 104. In another example, a separate tether may be used in order to anchor the various components of the rechargeable power supply 120 to the relevant part of the circulatory system and/or medical device 104.

The example embodiment relating to FIG. 1 has shown the rechargeable power supply 120 as a modular arrangement. In some implementations, it may be desirable to have an integrated arrangement.

In the example embodiment given above, the coil 111 and/or coil 152 may be concentrically wound coils (flat/planar coils), or helical coils (solenoid). In the case of flat coils, the position and/or orientation of the coils 111 and 152 may need to be changed so that the generated magnetic flux can be efficiently coupled.

A potential advantage of a helical coil may be that it is suitably wide enough to prevent it rotating out of its ‘y’ axis plane according to axis 170, when implanted within the vena cava 108 for example. Further, due to the direction of magnetic flux, the helical coil can freely rotate about the y axis within the vein without becoming misaligned. The size of the coil is such that it does not inhibit fluid flow within the circulatory system.

A potential advantage of a flat coil may be that it can be easily integrated on the power storage means, and that it takes up less area in the vein. However, this type of coil may require stabilising means in order to keep it in optimum alignment with generated magnetic flux from the extra-corporeal transmitting device.

An advantage of having a modular rechargeable power supply may be that only the implantable means for receiving power 110 needs to be correctly aligned. Thus the remaining components of the rechargeable power supply and the medical device can be aligned to suit the specific medical application, rather than to maximise coupling of magnetic flux.

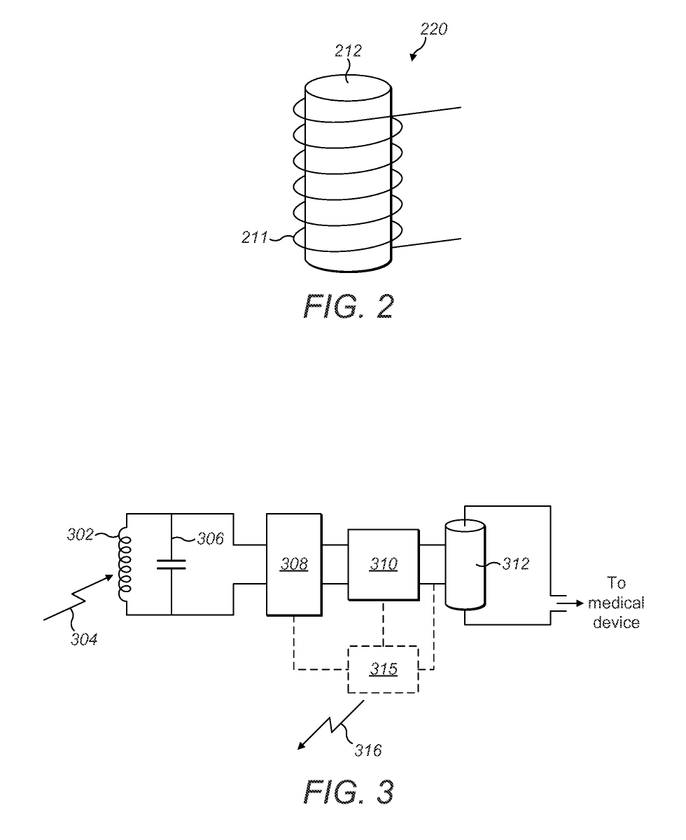

Referring to FIG. 2, a schematic representation of an integrated rechargeable power supply 220 is illustrated, comprising a power storage module 212 and a coil 211 that surrounds the power storage module 212. The integrated rechargeable power supply 220 may replace the rechargeable power supply 120 of FIG. 1. The operation of the integrated rechargeable power supply 220 is similar to the operation described in FIG. 1 for the rechargeable power supply 120. In this example embodiment, the coil 211 is arranged as a longitudinal coil (solenoid) that spirals along the entire length of the power storage module 212. It should be noted that the coil does not need to spiral along the entire length of the power storage module 212 to function. However, increasing the length of the spiral may have an advantage of increasing coupling area between the coil 211 and the coil 111 of the extra-corporeal power supply from FIG. 1.

In another example, the coil 211 may be formed from a number of solenoids that are coupled to the power storage module 212. This may have an advantage of improving robustness and efficiency of coupling in case one of the coils becomes damaged or misaligned.

Referring to FIG. 3, a simplified block diagram of a rechargeable power supply 300, for example, the rechargeable power supply 120/220 from FIGS. 1 and 2 is illustrated. In this example embodiment, the rechargeable power supply 300 comprises a means for wirelessly receiving power 302, for example a coil arranged to receiving magnetic flux 304 generated by an extra-corporal power supply (not shown), an optional capacitive element 306 coupled in parallel with the means for wirelessly receiving power 302, a means for supplying power 308, optional smoothing means 310, and power storage means 312. In this example embodiment, the various blocks may represent modular or integrated elements of the rechargeable power supply 300.

The means for wirelessly receiving power 302, in this example a coil, may be aligned such that it efficiently couples magnetic flux 304 generated from the extra-corporeal power supply, for example in the form of inductive coupling. The optional capacitive element 306 may be arranged to capacitively load the coil to form a tuned LC circuit. In such optional embodiments, the received magnetic flux may be generated from an extra-corporeal power supply also comprising a capacitively loaded tuned LC circuit, wherein both coils may be arranged to resonate at the same common frequency. This may have an advantage of increasing the magnetic coupling distance between the coils, increase efficiency of coupling, and reduce heat generated in the coils of the rechargeable power supply 300 and the extra-corporeal power supply. In some example embodiments, the common frequency may include frequencies that generate minimum heating of human body tissues. For example, the common frequency may be at least 1 MHz.

The means for wirelessly receiving power 302 may be operably coupled to the means for supplying power 308. The means for supplying power 308 receives AC power of a certain frequency and converts this AC power into DC power. In this example embodiment, the means for supplying power 308 may comprise either a full or a half wave rectifier, arranged to convert the AC power to DC power. The means for supplying power 308 may be optionally coupled to a smoothing means 310, which may be arranged to reduce voltage and/or current ripple before the resultant DC power is coupled to the power storage means 312. For example, smoothing means 310 may comprise additional capacitance and inductance in order to reduce ripple prior to the DC power being coupled to the power storage means.

In an optional embodiment, a control means 315 may be arranged and configured to control charging of the power storage means 312. The control means 315 may be coupled to the means for supplying power 308, the power storage means 312 and optionally the smoothing means 310. The control means 315 may determine the amount of charge stored in the power storage means 312 and regulate the means for supplying power 308 in order to efficiently charge and/or prevent overcharging of the power storage means 312. Optionally, the control means 315 may be arranged and configured to wirelessly transmit information via a wireless link 316 to the extra-corporeal power supply (not shown) to indicate if the power storage means 312 is fully charged. In an example, this may be achieved using a short range wireless interconnection, such as Bluetooth or near field communication (NFC). In an example, the control means 315 may comprise at least a processor, arranged to control the charging of the power storage means 312. The processor may also be arranged to prevent overcharging of the power storage means.

In another example, the wireless link 316 may be utilised to transmit diagnostic information to other extra-corporeal devices, or be utilised to update/change the operation of the rechargeable power supply 300 and/or an interconnected medical device. In another example, the wireless link 316 may be arranged to update software located in a memory within the control means 315 for controlling the processor.

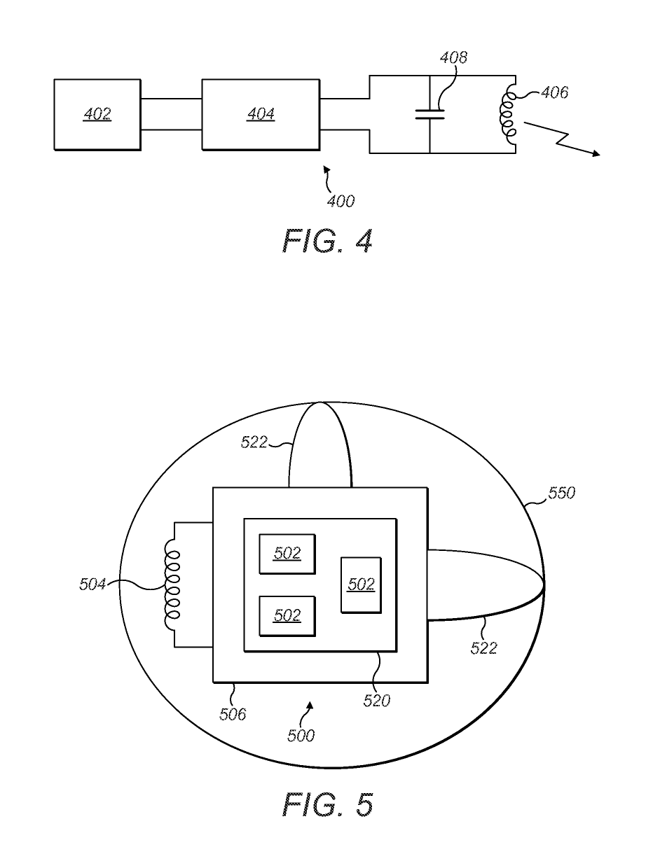

Referring to FIG. 4, a simplified block diagram of an extra-corporeal power transmitting device 400, for example, the extra-corporeal power transmitting device 150 from FIG. 1 is illustrated. In this example embodiment, the extra-corporeal power supply 400 comprises a power supply 402, arranged to supply either DC or AC power. The output of the power module 402 may be coupled to an optional means 404 for supplying the output power to means 406 for wirelessly transmitting power.

In an example where the power module 402 is configured to output DC power, the means 404 for supplying the output power may comprise a DC/AC converter, arranged to convert the DC power to AC power. The means 404 may supply an alternating current to the means 406, which may be a coil, to enable the means 406 to wirelessly transmit power. In this example, the AC current causes the coil to generate magnetic flux that is emitted by the means 406. An optional capacitive element 408 may be arranged in parallel with the means 406 in order to capacitively load the means 406 to form a capacitively loaded tuned LC circuit. The tuned LC circuit may resonate at a frequency substantially the same as a receiving coil of a rechargeable power supply implanted in the body of a patient, thereby allowing resonant inductive coupling.

In another example where the power module 402 is configured to output AC power, the means 404 may reduce the power via a passive device, such as a resistive network, and/or an active device, such as a switched mode power supply, in order to provide suitable AC power to the means 406. Optionally, the means 404 may comprise an AC/DC converter, such as for example a rectifier arrangement, that is configured to convert the received AC power into DC power, before reconverting to AC power via a DC/AC converter. An optional control module may be coupled to the AC/DC converter and the DC/AC converter in order to control the output power supplied to the means 406. Optionally, a capacitive element 408 may be arranged in parallel with the means 406, as discussed above.

During operation, the alternating current supplied to the means 406, may result in magnetic flux being produced. The means 406 may comprise a coil, which is configured to be positioned around the body of a patient, preferably the abdomen of the patient. In one example, the coil may be comprised within a positioning means, which may be substantially tubular. The patient may be able to slip the tubular positioning means around the abdomen, aligning the coil with an implanted medical device. In another example, the positioning means may be designed to be positioned around a limb of the patient, for example in the form of a bracelet. An implanted medical device may also be implanted within the limb in order to receive power from the bracelet shaped positioning means.

In a specific example, the power module 402 is arranged to supply mains power to the means 404, which comprises an AC-AC converter arranged to reduce the power to a level suitable for transmission by the means 406. The means 406 in this example is an electromagnetic coil that is, in use, positioned around the torso of a patient such the windings of the coil wrap around the torso of the patient. The electromagnetic coil is positioned such that it can efficiently couple power to a coil implanted within the patient's torso. In some cases the electromagnetic coil may comprise closely wound windings that approximates a planar coil. In other cases, the windings may be arranged such that they form a solenoid around the patient's torso, wherein the longitudinal axis of the solenoid is coaxially arranged with the longitudinal axis of the patient's torso.

Referring to FIG. 5, a schematic representation of an intra-corporeal medical device 500 comprising a rechargeable power supply is illustrated. In this example embodiment, the medical device 500 may comprise the rechargeable power supply 300 illustrated in FIG. 3. Furthermore, the medical device 500 may be implanted within a patient, for example implanted within the patient's heart 550. In other examples, the medical device 500 may be implanted within a patient's head or limbs.

The medical device 500 may comprise a number of application specific modules 502, and the rechargeable power supply. In this example embodiment, a coil 504 may be located at the exterior 506 of the medical device 500. This may have an advantage of increasing the coupling efficiency of the coil. The coil 504 may be covered with a medically-safe material such as silicon or latex in order to prevent corrosion of the coil 504. The application specific modules 502 and the remaining components of the rechargeable power supply may be arranged within a magnetic shield layer 520, arranged to protect the components of the medical device 500 from magnetic energy received by the coil 504 or other devices that may emit magnetic fields.

In an alternative example embodiment, the coil 504 may be positioned within the medical device 500, negating the need for covering the coil 504 in a medically-safe coating. The coil 504 may be positioned in a cavity situated between the exterior of the medical device 500 and the shield layer 520.

In this embodiment, the medical device 500 comprising the coil 504 may need to be aligned correctly with an extra-corporeal power supply (not shown) in order to maximise magnetic coupling. Stabilising means 522 may be optionally utilised on the medical device 500 in order to prevent the medical device from rotating/changing orientation within the patient. The coil 504 in this example is positioned perpendicular to the direction of fluid flow in the patient's heart 550. This may be so that the coil can be correctly aligned with the extra-corporeal power supply (not shown). Equally, the medical device 500 can be positioned such that the coil 504 is positioned parallel with the direction of fluid flow. The stabilising means 522 may be arranged to anchor the medical device 500 in a preferred orientation in order to maximise coupling between the coil 504 and the extra-corporeal power supply (not shown). The stabilising means 522 may comprise one or more anchors that are able to maintain the position of the medical device 500 without impeding fluid flow. For example, the stabilising means 522 may comprise one or more spring loaded securing arms that can be deployed once the medical device is in the correct position/orientation. The stabilising means 522 may also comprise a mesh, which is arranged to anchor the medical device 500 to the wall of the heart 550.

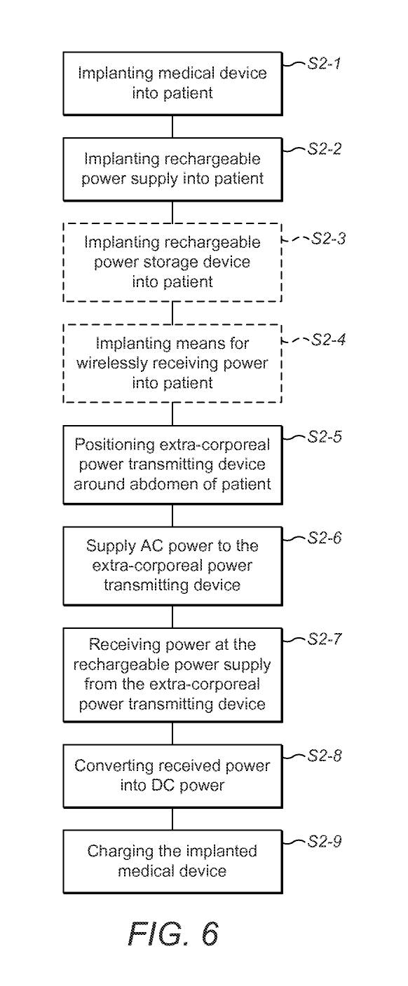

Referring to FIG. 6, a flow chart illustrating steps for supplying power to an intra-corporeal medical device, such as intra-corporeal medical device 500, is illustrated. At step S2-1, an implantable medical device, for example an LVAD, is implanted into a patient. The LVAD may be implanted via a percutaneous insertion device, and arranged between the patient's left atrium and aorta. At step S2-2, an implantable rechargeable power supply, for example the implantable power supply 300 from FIG. 3, is implanted in the patient. The rechargeable power supply may comprise modular components, in which case subsequent steps may be required to implant the rechargeable power supply. For example at step S2-3, a rechargeable power storage device, such as a rechargeable battery, is implanted into the patient and electrically connected to the medical device via an appropriate medically safe electrical connector. The rechargeable power storage device may be implanted in a different area of the patient's body compared to the medical device. For example, the rechargeable power storage device may be implanted in the inferior vena cava, and connected to the medical device via a suitable length connector. At step S2-4, a means for wirelessly receiving power, for example an electromagnetic coil, is implanted into the patient and electrically connected to the rechargeable power storage device. The coil may be implanted in a different area of the patient's body and electrically connected to the rechargeable power storage device via a suitable electrical connector. Positioning the coil in a different location to the medical device and/or the rechargeable power storage device may have an advantage of allowing optimum positioning of the medical device and/or the coil. For example, the coil may need to be implanted in a specific location in order to maximise energy transfer, which may be a different location and orientation to the medical device. At step S2-5, an extra-corporeal power transmitting device, for example the extra-corporeal power transmitting device 150 from FIG. 1, may be positioned around the abdomen of the patient. At step S2-6, AC power is supplied to the extra-corporeal power transmitting device, which is supplied to a coil surrounding the patient. The current in the coil generates a magnetic field, which may be at a medically safe resonant frequency that is transmitted to the implanted coil of the rechargeable power supply through the patient's body. At step S2-7, the implanted coil of the rechargeable power supply couples the generated magnetic field, in the form of magnetic flux. It should be noted that in some example implementations, the means for wirelessly receiving power may comprise a number of coils for receiving the transmitted field. These coils may be aligned differently with respect to each other in order to reduce coupling issues associated with alignment of these coils with the extra-corporeal power transmitting device. At step S2-8, the received magnetic flux is converted to DC power, via for example a suitable AC/DC converter, and supplied to the rechargeable power storage device. At step S2-9, the rechargeable storage device may receive the DC power and be appropriately charged, or the rechargeable power storage device may forward the DC power onto the medical device without charging the power storage device if, for example, the power storage device is fully charged.

An advantage of supplying power to the intra-corporal medical device in this way is that the rechargeable power supply may comprise a modular design. This may allow the device to be implanted in different areas of the patient's body, optimising the effectiveness of the device. Furthermore, the rechargeable power supply may be minimally invasive due to its size and modular design, which may allow it to be implanted in a similar manner to the medical device. Furthermore, the coil of the wireless receiving device may be elongated (a solenoid/helical coil) and aligned such that it substantially reduces magnetic coupling alignment issues. The coil may be formed from a number of coils arranged in different orientations with respect to the extra-corporeal transmitting device. This may reduce magnetic coupling alignment issues.

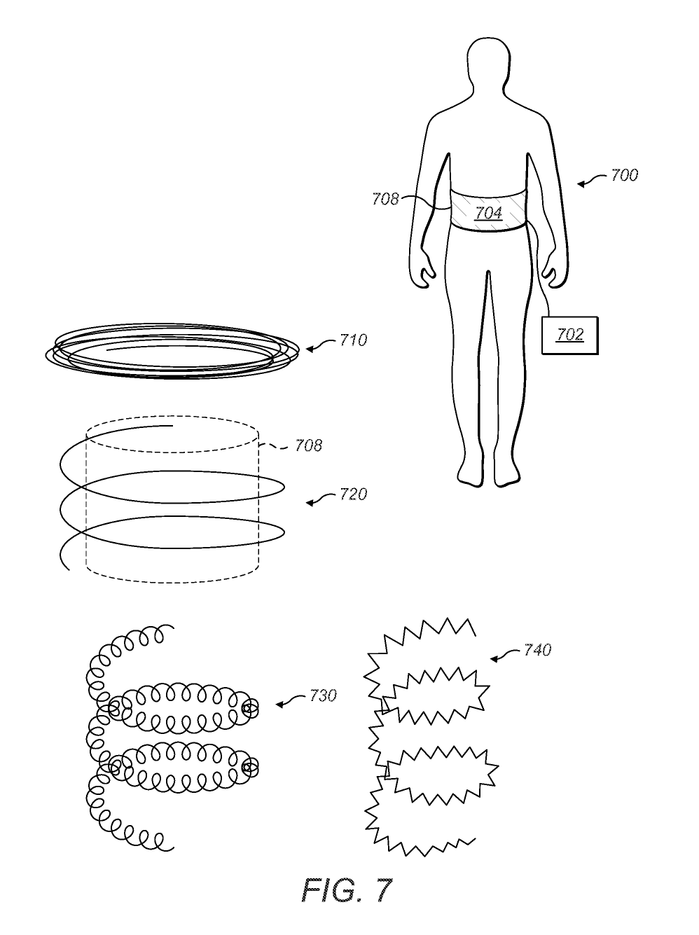

Referring to FIG. 7, a schematic representation of an extra-corporeal power transmitting device 700 for charging a rechargeable power supply, for example the rechargeable power supply 300 from FIG. 3, is illustrated. The extra-corporeal power supply 700 comprises a power supply 702, arranged to supply AC or DC power to a means for wirelessly transmitting power 704, via a suitable cable 706. The means for wirelessly transmitting power 704 is arranged and configured to transmit power to an implanted power receiving means of the rechargeable power supply 300 from FIG. 3. In this example embodiment, the means for wirelessly transmitting power 704 comprises an electromagnetic coil. The windings of the electromagnetic coil can be housed in a tubular positioning means 708. The tubular positioning means 708 may be arranged to form a hollow cylinder, wherein the hollow cylinder is positioned around the torso/abdomen of the patient, such that the longitudinal axis of the cylinder is coaxially aligned to the longitudinal direction of the patient's torso. In this embodiment, the windings of the electromagnetic coil are arranged and configured to be wound around the body of the hollow cylinder, such that the windings are coiled around the circumference of the cylinder. In one example, the windings of the coil may be arranged such that they have no longitudinal direction, as illustrated with respect to coil 710. In this case, the coil 710 needs to be positioned in close proximity to a respective power receiving device. In another example, the windings of the coil may form a helix, as illustrated by coil 720, which is arranged around the body of the tubular positioning means 708. Current applied to the helix, which can also be thought of as a solenoid, produces a magnetic filed that runs perpendicular to the orientation of the windings of the helix.

The tubular positioning means 708 may optionally be used to form a wearable garment that supports/houses the electromagnetic coil. Optionally, the electromagnetic coil may itself be formed from a continuous solenoid, as illustrated by coil 730 to form an extendible element. An advantage of this implementation is that the garment comprising the electromagnetic coil comprises elasticity, meaning that the garment may fit securely around a patient's abdomen, for example, without extra securing means being required. For example, the coil 730 may comprise spring like qualities, allowing it to be stretched around the patient's torso, within the garment, and subsequently attempt to return to its original shape thereby securing itself to the patient. In another example, the windings of the coil may be arranged as illustrated in coil 740, wherein the windings are bent to produce another coil with spring like qualities. The examples illustrated with respect to the coil 730 and coil 740 can be arranged in either a helical or planar arrangement as illustrated for coils 710 and 720.

In another example, the tubular positioning means 708 may be formed into a bracelet for wearing around an arm of a patient. This may be particularly advantageous if the rechargeable power supply 300 is positioned within the arm of the patient.

In another example, the tubular positioning means 708 may be formed into any suitable means for wearing around any part of the patient's body. As such, the rechargeable power supply 300 may also be located anywhere within the patient's circulatory system, depending on application.

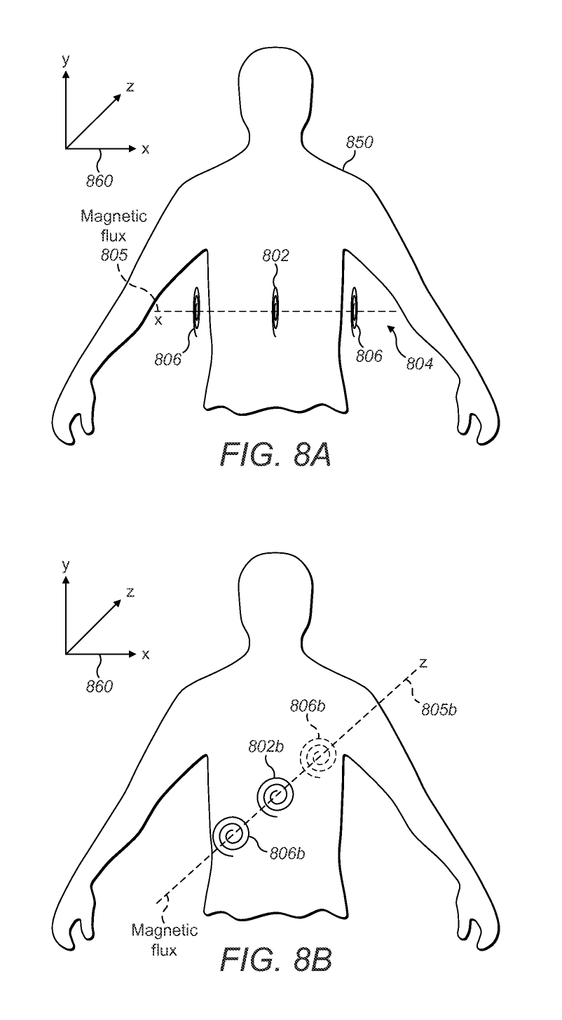

Referring to FIGS. 8a and 8b, an alternative schematic representation of a rechargeable power supply system for an intra-corporeal medical device implanted within the body of a patient is illustrated. For simplicity, the remainder of the rechargeable power supply has not been illustrated as its functionality is the same as that described for FIG. 1, unless stated otherwise with respect to FIGS. 8a and 8b. An axis 860 has been illustrated with respect to FIGS. 8a and 8b in order to help explain the direction of magnetic coupling.

Referring first to FIG. 8a, an implantable means 802 for wirelessly receiving power is situated within a patient 850. In this example embodiment, the implantable means 802 may be a planar coil or a helical coil/solenoid. An extra corporeal power transmitting device 804 may be positioned at a location on the patient's 850 abdomen, and comprise at least one means 806 for wirelessly transmitting power. In this example embodiment, means 806 for wirelessly transmitting power may comprise a planar coil that is aligned in such a way that magnetic flux 805 produced in this coil will propagate in the x direction according to axis 860. The implantable means 802 is aligned such that it can effectively couple the magnetic flux 805. In an example, the implantable means 802 is aligned co axially with the means 804 for wirelessly transmitting power along the x axis 860.

Although FIG. 8a has been illustrated with two means 804 for wirelessly transmitting power, it may be that only one means is required, if it is aligned so that magnetic flux can be coupled to the implanted means 802. However, situating at least two means 806 within the direction of magnetic flux may increase coupling efficiency compared to a single means.

Furthermore, implantable means 802 may comprise a number of coils with different alignments/orientations with respect to the direction of the generated magnetic flux. This may allow the implantable means 802 to still efficiently couple the transmitted magnetic flux even if one or more of the coils are not aligned in order to efficiently couple the transmitted magnetic flux.

FIG. 8b essentially operates in the same way as FIG. 8a. A difference being that the means 806b for wirelessly transmitting power have been arranged such that the magnetic flux 805b generated propagates in a z direction according to axis 860. Therefore, implanted means 802b is aligned to efficiently couple magnetic flux generated from means 806b. Again, means 806b may comprise one or more planar coils.

Optionally, the embodiments of FIGS. 8a and 8b can be combined. This may result in an implanted means 802 capable of receiving magnetic flux in an x and z direction, which may increase the coupling efficiency and may lessen the requirement for correct alignment in order to efficiently coupe magnetic flux from the means 806. In this embodiment, the dotted means 806b signifies that this means 806 is positioned on the back of the patient.

An advantage of the planar means 806 may be that they can be incorporated into a garment or simply positioned in a coupling position without the patient having to position the windings of the coils around their abdomen.

Although example embodiments of the invention have focussed on the medical device being implanted in the heart, it is envisaged that the medical device can be implanted at any required location in a patient, for example the patient's head, lungs, gastric system etc. An advantage of the modular design of the rechargeable power supply may be that in a specific example regarding the head, the coil could be situated away from the patient's brain, for example in the neck, thereby potentially reducing any undesirable heat generation from sensitive areas of the patient's body.

Furthermore, although example embodiments have focussed on the rechargeable power supply being situated within the circulatory system, this is not essential and the rechargeable power supply can be an integral part of the medical device, or be situated within the patient in a location other than in the circulatory system.

The power storage module 212/112 described in relation to FIG. 2 and FIG. 1 may be a form of rechargeable battery, for example an electrochemical storage battery comprised of known battery chemistries. Further, the power storage module may be a type of capacitor or super capacitor, which may be more tolerant to repeated charge and discharge cycles but require more frequent charge cycles.

Although already discussed above, it should be noted that the shape of the coil, for example a flat coil or a solenoid is not essential to implementing the invention. An effect of changing the coil may alter the direction of generated magnetic flux, requiring realignment of receiving and transmitting coils in order to effectively transfer power between an implantable rechargeable power supply and an extra-corporeal transmitting device.

Although some of the examples discussed above focus on the circulatory system with respect to the cardiovascular system, this is not intended to be limiting. It is envisaged that embodiments can equally be applied to any part of the circulatory system, for example within the limbs.

Embodiments discussed above may be utilised in any implantable device, whether subcutaneous or intravascular, for example, pacemakers, neural stimulators or heart pumps.

It should be implied from the above description that the sizes of the elements disclosed above may be changed to suit the application.

References to DC/AC converters and AC/DC converters may relate to any suitable device for carrying out aspects of the invention. For example, an AC/DC converter may comprise a rectifier network comprising a number of diodes. A DC/AC converter may comprise an inverter network comprising a number of semiconductor switches.

References to magnetic coupling, magnetic transmission/generation, or magnetic flux etc. can also be defined by the term ‘inductive coupling’.

Thus, from the above description, it can be seen that the present invention solves the problems described above.

1. A system for transmitting power to a rechargeable power supply, said rechargeable power supply comprising implantable power receiving means, and an implantable power storage means,
wherein said system comprises an extra corporeal power transmitting means arranged and configured to supply power to the rechargeable power supply by wirelessly transmitting power to the implantable power receiving means.

2. The system of claim 1, wherein the power transmitting means comprises an electromagnetic coil.

3. The system of claim 1, wherein the power transmitting means and the implantable power receiving means are configured to be magnetically coupled, thereby enabling power transmission between the extra-corporeal power transmitting means and the implantable power receiving means.

4. The system of claim 2, wherein the longitudinal axis of the electromagnetic coil is arranged and configured to be substantially parallel to the longitudinal axis of an implantable power receiving means.

5. The system of claim 1, further comprising positioning means for positioning the power transmitting means relative to the implantable power receiving means to enable magnetic coupling.

6. The system of claim 5, wherein the positioning means, in use, is positioned around the abdomen of a patient.

7. The system of claim 5, wherein the positioning means is substantially tubular.

8. The system of claim 7, wherein the positioning means is a wearable garment.

9. The system of claim 7, wherein the positioning means is a vest or a belt.

10. The system of claim 5, wherein the power transmitting means and/or the positioning means are each or both made from an extendible material.

11. A method for supplying power to an intra-corporeal medical device in a system according to any one of claims 1-10, comprising the step of implanting the rechargeable power supply in a patient.

12. The method of claim 11, further comprising the step of wirelessly transmitting power from the extra-corporeal power transmitting device to the implantable power receiving means.

13. The method of claim 12, further comprising the step of supplying the power received by the power receiving means to the power storage means.

14. The method of claim 11, wherein the power receiving means and/or the power storage means are, in use, positioned in the circulatory system, in a vein or an artery.

15. The method of claim 14, wherein the power receiving means and/or the power storage means are, in use, positioned within the inferior vena cava.
